(19) United States
(12) Patent Application Publication
(10) Pub. No.: US 2005/0288634 A1
(43) Pub. Date: Dec. 29, 2005

(54) Universal seal

(76) Inventors: Peter T. O'Heeron, Houston, TX (US); Ivan W. Byford, Houston, TX (US)

Correspondence Address: Clarence E. Eriksen;Jackson Walker L.L.P., Suite 2100, 112 E. Pecan Street, San Antonio, TX, 78205-1521, US

(21) Appl. No.: 10/878,832

(22) Filed: Jun. 28, 2004

(51) Int. Cl. A61M 5/178

(52) U.S. Cl. 604/167.06

(57) ABSTRACT

A seal is disclosed for installation on a port of a medical device which preferably is a trocar. A seal in accordance with present invention has a first portion which has a bore and a first outer wall of a first thickness. The base of the first portion engages the port. A seal in accordance with the present invention has a second portion with a top and a passage through the top. The second portion has a second outer wall which is integral with the first outer wall and that has a thickness at its juncture with the first outer wall that is less than the thickness of the first outer wall.

### BACKGROUND OF THE INVENTION

1. Field of the Invention

The present invention relates to surgical instruments known as trocars which are used in endoscopic surgery to pierce or puncture an anatomical cavity to provide communication with the cavity during a surgical procedure. More particularly, the present invention relates to a seal to prevent the escape of insufflated gas during the performance of surgical procedures using the trocar.

2. Description of the Prior Art

Endoscopic surgery is an essential method of performing surgical operations and has become the surgical procedure of choice, because of its patient care advantages over “open surgery.” One form of endoscopic surgery is laparoscopic surgery, and a significant advantage of laparoscopic surgery over open surgery is the decreased post-operative recovery time. In most instances, a patient is able to leave the hospital within hours after laparoscopic surgery has been performed. With open surgery, several days of hospital care may be required before the patient is discharged. Additionally, laparoscopic surgery achieves decreased incidents of post-operative abdominal adhesions, decreased post-operative pain, and enhanced cosmetic results.

Conventionally, a laparoscopic surgical procedure begins with the insufflation of the abdominal cavity with carbon dioxide. The introduction of this gas into the abdominal cavity lifts the abdominal wall away from the internal viscera. The abdominal wall is then penetrated with a device known as a trocar, which includes a housing assembly, a cannula assembly attached to the housing assembly to form a bore through the trocar, and a piercing element called an obturator. The obturator slides through an access port formed on the upper (i.e. proximal) end of the housing assembly and through the bore of the trocar. The obturator has a diameter which is substantially the same as the diameter of the access port. After insertion of the trocar through the abdominal wall of the patient, the obturator is removed by the surgeon while leaving the cannula or tube protruding through the body wall. Laparoscopic instruments can then be inserted through the cannula to view internal organs and to perform surgical procedures.

Once the obturator is removed from the bore of the housing, it is necessary to obstruct the access port so that the carbon dioxide gas introduced into the abdominal cavity of the patient is contained. Traditionally, a trocar includes a spring-loaded flapper valve which opens when the obturator is inserted and which closes when the obturator is removed from the cannula to keep the insufflated gas from escaping. However, the insertion of laparoscopic instruments into the trocar re-opens the flapper door. To prevent escape of the insufflated gas upon insertion of a laparoscopic instrument, a trocar also comprises a seal which is capable of providing sealing for laparoscopic instruments having varying diameters e.g. between 5 mm and 12 mm. Since such seals are capable of providing sealing during the same surgical procedure for laparoscopic instruments of varying diameters, they are commonly referred to as “universal” seals.

Various designs of universal seals have been proposed. See for example, U.S. Pat. Nos. 5,350,364; 5,385,553; 5,407,433; 5,512,053; 5,628,732, 5,827,228; 5,342,315; and 4,112,932. Such prior art seals comprise a plurality of mechanical parts which must be assembled and are usually expensive.

### SUMMARY OF THE INVENTION

In accordance with the present invention, a seal is provided for external attachment to an access port of a medical device. In a preferred embodiment, a seal in accordance with the present invention is for external attachment to an access port which is located at the proximal end of a trocar.

A seal in accordance with the present invention comprises a first portion which has a bore through it. The first portion has a first outer wall with a first thickness and has a base which is formed for engagement with the access port at the proximal end of the trocar. Preferably, the first portion of the seal is generally tubular in shape.

A seal in accordance with the present invention further comprises a second portion which has a top with a passage through the top that is aligned with the bore. The second portion has a second outer wall which is integral with the first outer wall. The thickness of the second outer wall at its juncture with the first outer wall is less than the thickness of the first outer wall.

In a preferred embodiment, the top of the seal is generally circular in shape and has plurality of folds formed therein. In one embodiment of a seal in accordance with the present invention, the folds comprise a plurality of spiral-like folds which are formed around the passage. In yet another embodiment of the present invention, the folds comprise a plurality of circular folds each of which is concentric with the passage. In yet a third embodiment, the folds comprise a plurality of substantially straight, accordion-like folds which are arranged in a generally circular pattern around the passage.

In each of these embodiments, the normal diameter of passage is approximately 2 millimeters. The passage is expandable to a diameter of about 13 millimeters upon insertion of a surgical instrument through the passage. Thus, a seal in accordance with the present invention functions to seal on surgical instruments having diameters between 2 and 13 millimeters, while allowing the surgeon to move the surgical instrument in any plane without loss of sealing function. Upon removal of the surgical instrument from the trocar, the passage returns to its normal diameter of 2 millimeters due to the memory characteristics of the seal material. A seal in accordance with the present invention allows a surgeon to use varying diameter surgical instruments during a surgical procedure and to move each such instrument in any direction without loss of the sealing function between the seal and the instrument.

In a preferred embodiment of the present invention, the second portion of the seal is generally shaped like a frustum of a cone with a base and a conical outer wall having a second wall thickness. The base of the frustum forms the top of the seal, and the top of the seal has a generally circular shape.

### BRIEF DESCRIPTION OF THE DRAWINGS

FIG. 1 is an exploded perspective view of an exemplary embodiment of a trocar.

FIG. 2 is a lateral section view of the proximal end of the body of the trocar shown in FIG. 1.

FIG. 3 is a perspective view of one embodiment of a seal in accordance with the present invention.

FIG. 4 is a lateral section view of the proximal end of the trocar illustrated in FIG. 1 with the seal of FIG. 3 installed on the proximal end.

FIG. 5 is a lateral section view of the apparatus of FIG. 4 with a medical instrument installed through the trocar.

FIG. 6 is a lateral section view of the apparatus illustrated in FIG. 3 with a medical instrument being withdrawn from the trocar.

FIG. 7 is a perspective view of another embodiment of a seal in accordance with the present invention.

FIG. 8 is an enlarged perspective view of the accordion like folds shown in FIG. 7.

FIG. 9 is a lateral section view of the proximal end of the body of the trocar shown in FIG. 1 with a seal as illustrated in FIG. 7 installed on the proximal end of the trocar.

### DESCRIPTION OF THE PREFERRED EMBODIMENTS

It will be appreciated that the present invention can take many forms and embodiments. Certain embodiments are described herein to give an understanding of the invention. The embodiments described herein are intended to illustrate and not to limit the invention.

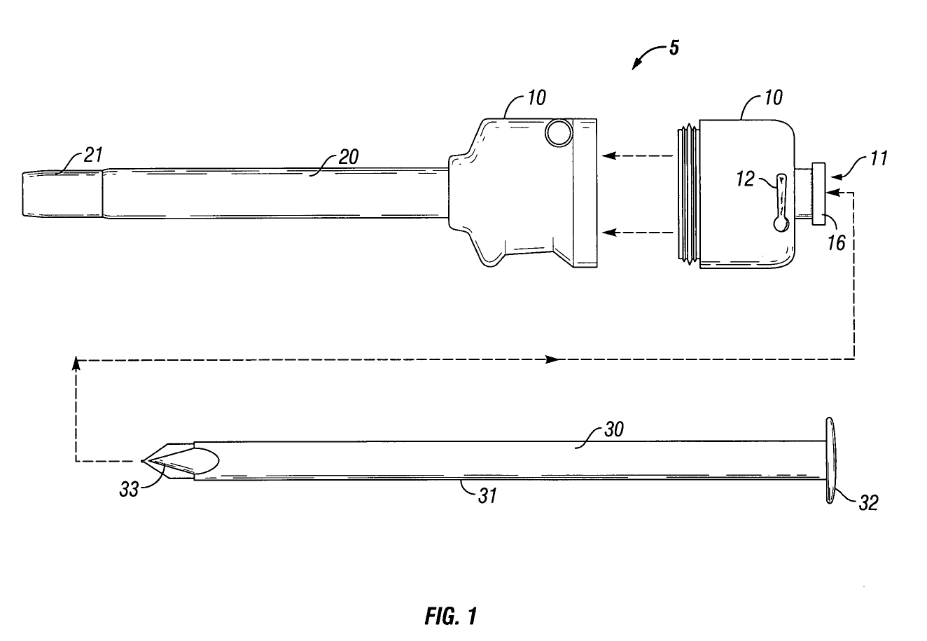

With reference to FIG. 1, an embodiment of a trocar 5 comprises a housing assembly 10 to which is attached a cannula assembly 20. The cannula assembly 20 is a hollow tube, and when attached to the housing assembly 10, a bore is defined through the trocar 5. An access port 11 is formed in the proximal end of the housing assembly 10 such that the access port 11 and the bore defined by the cannula assembly 20 are axially aligned. The diameter of the access port 11 may, for example, be between 2 mm and 22 mm.

Still with reference to FIG. 1, the trocar 5 also includes an obturator assembly 30 having a shaft 31 with an arcuate-shaped cap 32 at the proximal end of the shaft and a piercing tip 33 at the distal end of the shaft. The obturator assembly 30 has a diameter substantially the same as the diameter of the access port, and the obturator is inserted into the housing assembly 10 through the access port 11. The obturator slides in the bore that is defined by the combination of housing assembly 10 and cannula assembly 20. The trocar 5 may comprise a safety shield 21, although the present invention is not limited to seals for trocars with safety shields.

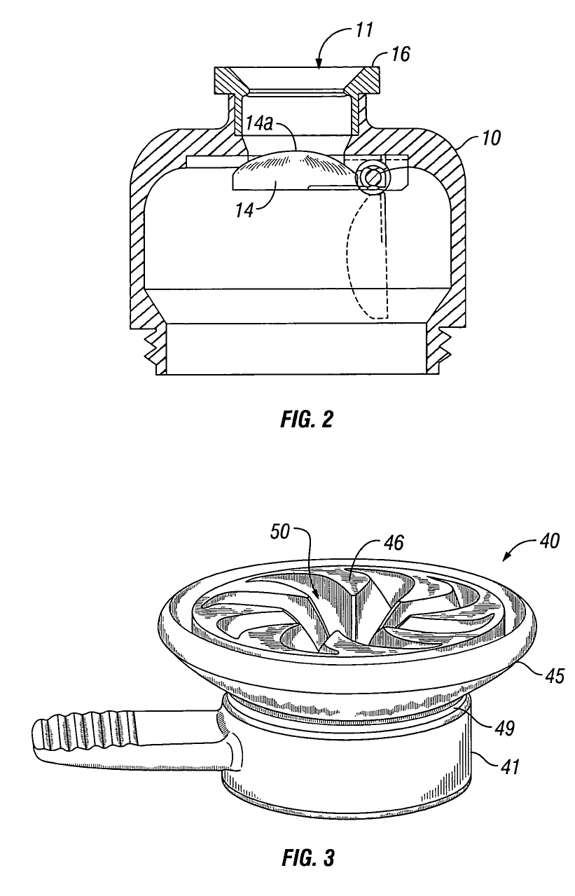

With reference to FIG. 2, the trocar 5 includes a flapper valve door 14 for regulating communication through the access port 11. As illustrated in FIG. 2, the flapper valve door 14 may have a domed protrusion 14A which engages the housing 10 to form a seal when the flapper valve door 14 is closed. The flapper valve door 14 is rotatably connected to the housing assembly 10, and the flapper valve door 14 is rotatable between a closed position and an open position, as shown by the dotted lines in FIG. 2. Resistance mechanisms, such as torsion springs or compression springs (not shown), may be used to bias the flapper valve door 14 in the closed position. A manual flapper door actuator 12 is provided for manual rotation of the flapper valve door 14 between the closed position and range of open positions. Withdrawal of the obturator assembly 30 from the housing assembly 10 results in the closure of the flapper valve door 14.

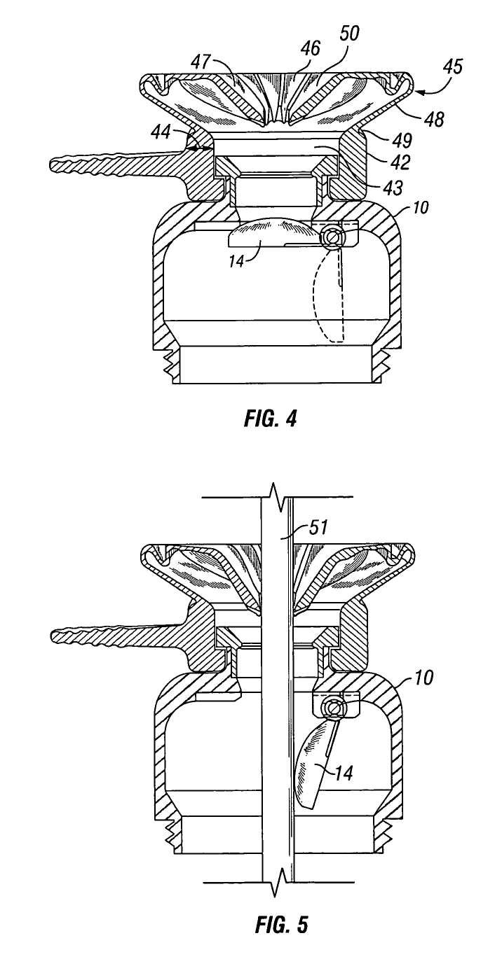

After the obturator assembly 30 is withdrawn from the trocar 5, a seal in accordance with the present invention is attached to access port 11. With reference now to FIGS. 3 and 4, a first embodiment of a seal in accordance with the present invention is illustrated. Seal 40 comprises a first portion 41 having a bore 43 and having a base that is formed for engagement with the flange 16 at access port 11 at the proximal end of the trocar. The first portion 41 of seal 40 has a first outer wall 42 having a wall thickness 44. Preferably, first portion 41 is generally tubular in shape.

Still referring to FIGS. 3 and 4, seal 40 further comprises an second portion 45 which has a top 46 with a passage 47 formed therein, which passage is aligned with bore 43. The second portion 41 has an outer wall 48 which is integral with outer wall 42. The juncture between the first outer wall 42 and second outer wall 48 is designated as 49. The thickness of outer wall 48 at the juncture 49 is less than the wall thickness of the first outer wall 42.

Preferably, the second portion 45 of seal 40 has a plurality of folds 50 formed in the top 46. In the embodiment illustrated in FIGS. 3 and 4, the folds 50 are spiral-like in shape and extend downwardly from the top 46 to form passage 47.

The normal diameter of passage 47 is approximately 2 millimeters. Passage 47 is, however, expandable from the 2 millimeter diameter to a diameter of about 13 millimeters, and is thus able to accommodate surgical instruments of varying diameters between 2 and 13 millimeters without loss of sealing function. Upon removal of the surgical instrument from the trocar, the passage 47 returns to its normal 2 millimeter diameter due to the memory characteristics of the material from which the seal is fabricated.

With reference to FIG. 5, there is illustrated the seal and trocar body configuration of FIG. 4 with a surgical instrument 51 inserted into the trocar. The surgical instrument 51 passes through the seal 30 and opens flapper door 14 as it enters the body of the trocar. The diameter of surgical instrument 51 determines the extent to which the folds 50 will open as the surgical instrument is inserted.

As a result of the thickness of the outer wall 48 being less than the thickness of outer wall 42 at location 49, the second portion 45 of the seal 40 is able to move or pivot with respect to the first portion 41 when a surgical instrument 51 is inserted into the trocar. Surgical instrument 51 may move in any direction without loss of sealing function.

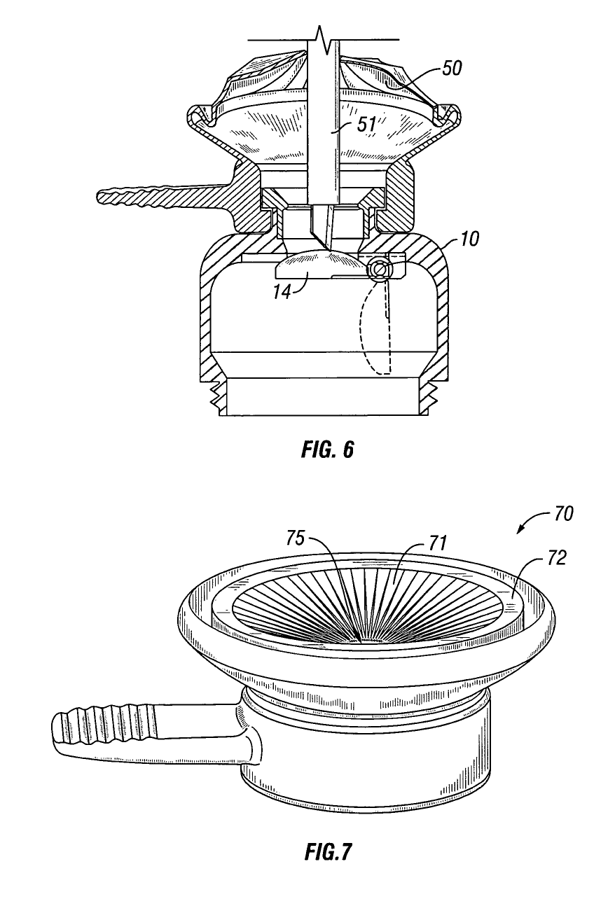

With reference now to FIG. 6, there is illustrated the seal of 40 of FIGS. 3 and 4 during removal of the medical instrument 51 from the trocar. When removal occurs, the folds 50 continue to provide a sealing function for the surgical instrument 51 because the folds 50 extend upwardly as the surgical instrument 51 is withdrawn from the seal. After the medical instrument 50 is completely withdrawn from the seal, the seal returns to its normal state as shown in FIGS. 3 and 4.

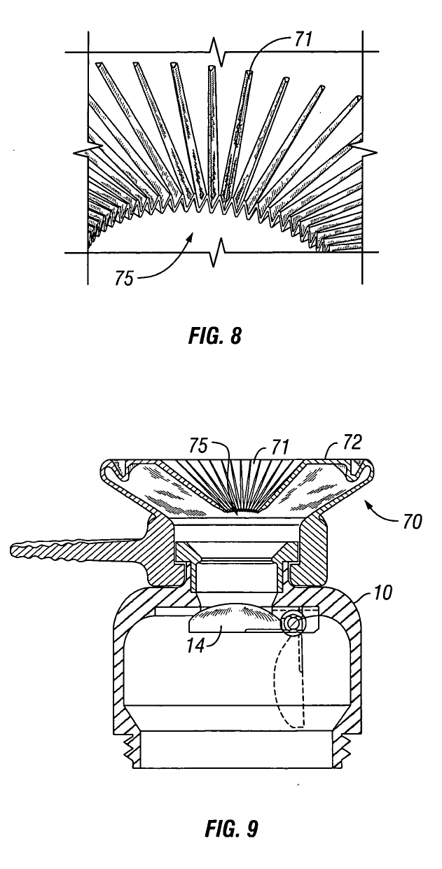

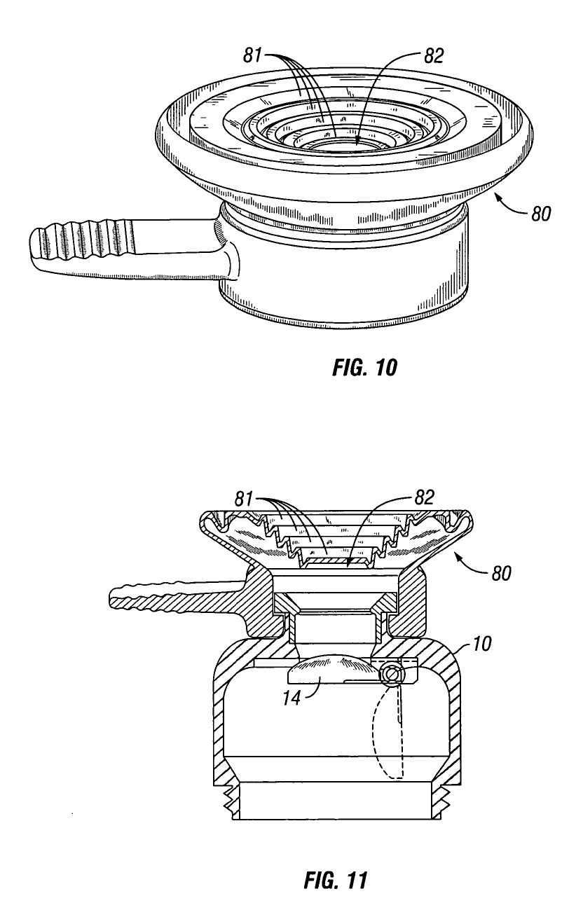

With reference now to FIG. 7, there is illustrated another embodiment of a seal 70 in accordance with the present invention. The structure and characteristics of seal 70 are identical to the structure and characteristics of seal 40 as illustrated in FIGS. 3 and 4, with the exception that seal 70 includes a plurality of accordion-like folds 71 which are formed in the top 72 a generally circular pattern and which extend downwardly to form a passage 75 as illustrated in FIGS. 7, 8 and 9.

With reference now to FIGS. 10 and 11, another embodiment of a seal in accordance with the present invention is illustrated. The structure and characteristics of seal 80 are also identical to the structure and characteristics of seal 40 shown in FIGS. 3 and 4, with the exception that in the embodiment in FIGS. 10 and 11, the top comprises a plurality of circular folds and each circular fold 81 is concentric with the passage 82 in the seal.

A seal in accordance with the present invention may be fabricated from any pliable material that is FDA approved and that has memory characteristics. Preferably, seals in accordance with the present invention are fabricated from Norel brand silicone.

1 A seal for external attachment to a port of a medical device, comprising:
(a) a first portion with a bore, the first portion having a first outer wall of a first thickness and having a base which is formed for engagement with the port; and
(b) a second portion having a top with a passage through the top which is aligned with the bore and having a second outer wall which is integral with the first outer wall , said second outer wall having a thickness at the juncture of the first and second outer walls that is less than the thickness of the first outer wall.

2. The seal of claim 1, wherein the seal is for attachment to an access port at the proximal end of a trocar.

3. The seal of claim 1, wherein the first portion is generally tubular in shape.

4. The seal of claim 1, wherein the top comprises a plurality of spiral-like folds formed therein around said passage.

5. The seal of claim 4, wherein the normal diameter of passage in the seal is approximately 2.0 millimeters.

6. The seal of claim 5, wherein the diameter of the passage in the seal is expandable from approximately 2.0 millimeters to about thirteen millimeters without loss of sealing function.

7. The seal of claim 4, wherein it comprises a pliable material.

8. The seal of claim 7, wherein the pliable material is silicone.

9. The seal of claim 1, wherein the top comprises a plurality of circular folds formed therein, where each of the circular folds is concentric with the passage.

10. The seal of claim 9, wherein the normal diameter of the passage is approximately 2.0 millimeters.

11. The seal of claim 10, wherein the diameter of the passage is expandable from approximately 2.0 millimeters to about thirteen millimeters without loss of sealing function.

12. The seal of claim 11, wherein it comprises a pliable material.

13. The seal of claim 12, wherein the pliable material is silicone.

14. The seal of claim 1, wherein the top comprises a plurality of substantially straight, accordion-like folds which are arranged in a generally circular pattern around passage.

15. The seal of claim 14, wherein the normal diameter of the passage is approximately 2.0 millimeters.

16. The seal of claim 15, wherein the diameter of the passage is expandable from approximately 2.0 millimeters to about thirteen millimeters without loss of sealing function.

17. The seal of claim 14, wherein it comprises a pliable material.

18. The seal of claim 17, wherein the pliable material is silicone.

19. A seal for external attachment to the access port of a trocar, comprising:
(a) a first portion with a bore, the first portion having a first outer wall of a first thickness and having a base formed for engagement with the access port; and
(b) a second portion having a top and a passage through the top with folds formed in the top around the passage and having a second outer wall which is integral with the first outer wall, said second outer wall having a thickness at the juncture of the first and second outer walls that is less than the thickness of the first outer wall.

20. The seal of claim 19, wherein the folds comprise a plurality of spiral folds.

21. The seal of claim 19, wherein the folds comprise a plurality of accordion-like folds.

22. The seal of claim 19, wherein the folds comprise a plurality of circular folds where each of said circular folds is concentric with the passage.

23. A seal for external attachment to an access port at the proximal end of a trocar, comprising:
(a) a lower first portion which is generally tubular in shape with a bore and having a first outer wall with a first thickness and having a base which is formed for engagement with the access port;
(b) a second portion having a top which is generally circular in shape and having folds formed therein that extend downwardly from the top of the seal to form a passage through the top which is aligned with the bore and having a second outer wall which is integral with the first outer wall, where the second outer wall having a thickness at the juncture between the first and second outer walls that is less than the thickness of the first outer wall.

24. The seal of claim 23, wherein the folds comprise a plurality of spiral-like folds which expand downwardly from the top of the seal to the passage.

25. The seal of claim 24, wherein the normal diameter of the passage is approximately 2.0 millimeters.

26. The seal of claim 25, wherein the diameter of the passage is expandable from approximately 2.0 millimeters to about thirteen millimeters without loss of sealing function.

27. The seal of claim 24, wherein it comprises a pliable material.

28. The seal of claim 27, wherein the pliable material is silicone.

29. The seal of claim 23, wherein the folds comprise a plurality of circular folds, where each of said folds is concentric with the passage.

30. The seal of claim 29, wherein the normal diameter of the passage is approximately 2.0 millimeters.

31. The seal of claim 30, wherein the diameter of the passage is expandable from approximately 2.0 millimeters to about thirteen millimeters without loss of sealing function.

32. The seal of claim 31, wherein it comprises a pliable material.

33. The seal of claim 32, wherein the pliable material is silicone.

34. The seal of claim 33, wherein the folds comprise a plurality of substantially straight, accordion-like folds which extend downwardly from the top of the seal to the passage.

35. The seal of claim 34, wherein the normal diameter of the passage is approximately 2.0 millimeters.

36. The seal of claim 35, wherein the diameter of the passage is expandable from approximately 2.0 millimeters to about thirteen millimeters without loss of sealing function.

37. The seal of claim 34, wherein it comprises a pliable material.

38. The seal of claim 37, wherein the pliable material is silicone.
